(19) United States
(12) Patent Application Publication
(10) Pub. No.: US 2013/0023575 A1
(43) Pub. Date: Jan. 24, 2013

(54) COMPOSITIONS AND METHODS FOR THE TREATMENT OF OCULAR SURFACE ALLERGIES

(71) Applicants: Kamran Hosseini, Hayward, CA (US); Lyle Bowman, Pleasanton, CA (US)

(72) Inventors: Kamran Hosseini, Hayward, CA (US); Lyle Bowman, Pleasanton, CA (US)

(21) Appl. No.: 13/188,730

(22) Filed: Jul. 22, 2011

(51) Int. Cl. A61K 31/196; A61P 27/14; A61P 9/08; A61K 31/4174

(52) U.S. Cl. 514/401; 514/567

(57) ABSTRACT

The disclosure provides ophthalmic compositions, systems and methods for the treatment of ocular surface allergies. More particularly the present invention relates to compositions of bromfenac for the treatment of the signs and symptoms of ocular surface allergies due to seasonal allergies.

The present invention relates to ophthalmic compositions; more particularly to ophthalmic compositions of nonsteroidal anti-inflammatory agents for the treatment of ocular surface allergies. More particularly the present invention relates to low dosage compositions of bromfenac for the treatment of the signs and symptoms of ocular surface allergies including those signs and symptoms due to seasonal allergens.

### BACKGROUND OF THE INVENTION

A variety of factors are important in topical administration of medicaments to the eye to treat ocular surface allergies, among them: comfort, control, consistency and accuracy of dosage, type and time of any vision interference, ease of administration, number of times a dosage must be administered and timing of delivery. Prior ophthalmic delivery systems for the treatment of ocular surface allergies have suffered drawbacks in one or more of those areas.

### SUMMARY OF THE INVENTION

The invention provides a novel composition of low dosage bromfenac for the treatment of ocular surface allergies that can overcome any of the drawbacks discussed above.

The present invention provides methods of treating ocular surface allergies including those signs and symptoms due to seasonal allergens with topical ophthalmic formulations containing a low dosage of bromfenac (a non-steroidal anti-inflammatory agent), and a flowable mucoadhesive polymer.

In one aspect, the present invention provides an ophthalmic composition including bromfenac in an amount of 0.01% to 0.05% by weight of the composition and a flowable mucoadhesive polymer. In certain embodiments bromfenac is present in an amount of from 0.01% to 0.04% by weight of the composition. The composition has a viscosity formulated for administration to the eye of a mammal in drop form. In another related embodiment, bromfenac is retained in or carried with the flowable mucoadhesive polymer. In another related embodiment, the flowable mucoadhesive polymer is a sustained release delivery system. In another related embodiment, the flowable mucoadhesive polymer is a carboxy-containing polymer, such as polycarbophil or DuraSite®.

In another related embodiment, the mucoadhesive polymer is in an amount of about 0.5% to about 1.5% by weight of the composition. In another related embodiment, the polymer is in an amount of about 0.8% to about 1.0% by weight of the composition. In another related embodiment, bromfenac is in an amount of about 0.01% to about 0.05% by weight of the composition. In another related embodiment, the composition has a pH of about 8.0 to about 8.6. In another related embodiment, the composition has a pH of about 8.3. In another related embodiment, the viscosity of the composition is in the range of about 1,000 to about 2,000 centipoises (cps). In another related embodiment, the viscosity of the composition is about 1,500 cps.

In another aspect, the invention relates to a sustained release bromfenac delivery system, including a flowable mucoadhesive polymer and a therapeutically effective amount of bromfenac in an ophthalmic composition; wherein the flowable mucoadhesive polymer is in an amount of about 0.5% to about 1.5% by weight of the composition and bromfenac is in an amount of about 0.01% to about 0.04% by weight of the composition.

In another aspect, the invention relates to a method of treating ocular surface allergies including those seasonal allergies, comprising a step of providing to the eye, an ophthalmic composition comprising bromfenac in an amount of about 0.01% to about 0.05% by weight of the composition and a flowable mucoadhesive polymer in an amount of about 0.5% to about 1.5% by weight of the composition. In certain embodiments of this aspect of the invention, the composition contains from about 0.01% to about 0.04% bromfenac. The method also includes a step of administering the composition to the eye of a mammal in need thereof to treat ocular surface allergies. In another related embodiment, the ocular surface allergy is caused by a seasonal allergen such as pollen, ragweed, trees, grass, and mold.

It is to be understood that both the foregoing general description and the following detailed description are exemplary and explanatory only and are not restrictive of the invention, as claimed.

### DETAILED DESCRIPTION OF THE INVENTION

The present invention generally relates to the treatment of ocular surface allergies including those ocular surface allergies due to seasonal allergens and other allergens such as animal dander and animal hair. As used herein, the term “ocular seasonal allergies” means those symptoms and signs of allergic conjunctivitis due to an allergen. Topical ophthalmic formulations containing a low dose of bromfenac, a non-steroidal anti-inflammatory agent (NSAID), and a flowable mucoadhesive polymer are used. Not only have the applicants found that the method effectively treats ocular surface allergies including the treatment of the mild signs, they have also unexpectedly discovered that a low dosage of bromfenac effectively treats these symptoms.

Allergic reactions that affect the conjunctiva, which covers the surface of the eye are commonly referred to as allergic conjunctivitis. Allergic conjunctivitis is divided into several major subtypes, but the most common subtypes are seasonal allergic conjunctivitis (SAC) and perennial allergic conjunctivitis (PAC). SAC and PAC are triggered by an immune reaction involving a sensitized individual and an allergen. Symptoms of SAC and PAC include mild ocular itching, and tearing. Stronger symptoms of allergic conjunctivitis include ocular hyperemia, chemosis, lid swelling, and exudation of fluid from blood vessels into surrounding tissue, which in turn leads to inflammation and possible tissue damage. The present invention relates to the treatment of the mild signs and symptoms of allergic conjunctivitis due to allergens such as pollen, ragweed, trees, grass, and mold, animal hair and animal dander, and more particularly to seasonal allergens such as pollen, ragweed, trees, grass, and mold.

The increased absorption and retention of a low dosage of bromfenac in the eye, provided by the compositions of the present invention, allow subjects to treat ocular surface allergies with fewer applications of the composition. A higher absorption and retention of the low dosage of bromfenac in the eye improves comfort, control, consistency and accuracy of dosage, type and time of any vision interference, ease of administration, and timing of delivery.

Bromfenac is a non-steroidal anti-inflammatory agent commonly used to treat patients who have undergone cataract removal. The chemical structure of bromfenac is disclosed in U.S. Pat. No. 4,910,225. A sterile ophthalmic solution of bromfenac as sodium salt equivalent to 0.09% bromfenac free acid is currently marketed as Bromday® by ISTA/Senju Pharmaceuticals with a recommended dosing schedule of 1 drop per 24 hours. However, Bromday® has been shown not to provide a good control of prostaglandin-mediated inflammation when compared to ketorolac (another NSAID) partly due to its concentration drop in the eye after twenty four hours, consistent with its on-label dosing schedule (Bucci et al., J Cataract Refract Surg. 34(9):1509-12 (2008)).

The present methods and compositions provide novel topical ophthalmic formulations containing low dosage bromfenac and a flowable mucoadhesive polymer for the treatment of signs and symptoms of ocular surface allergies.

As used herein the term “ophthalmic composition” refers to a composition intended for application to the eye or its related or surrounding tissues such as, for example, eyelid. The term also includes compositions intended to therapeutically treat conditions of the eye itself or the tissues surrounding the eye and compositions administered via the ophthalmic route to treat therapeutically a local condition other than that involving the eye. The ophthalmic composition can be applied topically or by other techniques, known to persons skilled in the art, such as injection to the eye or its related tissues. Examples of suitable topical administration to the eye include administration in eye drops and by spray formulations. A further suitable topical administration route is by subconjunctival injection. The agents can also be provided to the eye periocularly or retro-orbitally. Although it is an advantage of the invention that intracameral administration is not required, this and other routes of administration are not outside the scope of the invention.

As used herein the term “flowable mucoadhesive polymer” refers to a carboxy-containing polymer, e.g., lightly crosslinked polymers of acrylic acid or the like, having an optimal in vivo mucosal absorption rate, safety, degradability and flowability for an eye drop. The flowable mucoadhesive polymers used in the present invention are water insoluble, water-swellable, biodegradable polymer carriers including lightly crosslinked carboxy-containing polymers such as polycarbophil (Noveon® AA-1, Lubizol Corp., Wickliffe, Ohio) or other Carbopol® polymers (Lubizol Corp., Wickliffe, Ohio). Suitable carboxy-containing polymers for use in the present invention and methods for making them are described in U.S. Pat. No. 5,192,535 to Davis et al. A suitable carboxy-containing polymer system for use in the present invention is known by the tradename DuraSite® (InSite Vision Inc., Alameda, Calif.), containing polycarbophil, which is a sustained release topical ophthalmic delivery system that releases drug at a controlled rate. DuraSite® encompass lightly crosslinked polymers that are prepared by suspension or emulsion polymerizing at least about 90% by weight of a carboxyl-containing monoethylenically unsaturated monomer such as acrylic acid with from about 0.1% to about 5% by weight of a polyfunctional, or difunctional, crosslinking agent such as divinyl glycol (3,4-dihydroxy-1,5-hexadiene), having a particle size of not more than about 50 μm in equivalent spherical diameter, when formulated with an ophthalmic medicament, e.g., bromfenac, into solutions or suspensions in aqueous medium in which the amount of polymer ranges from about 0.5% to about 1.5% by weight, based on the total weight of the aqueous suspension, the pH is from about 7.4 to about 8.5, and in some embodiments, about pH 8.3, and the osmotic pressure (osmolality or tonicity) is from about 10 mOsM to about 400 mOsM, provide new topical ophthalmic medicament delivery systems having suitably low viscosities which permit them to be easily administered to the eye in drop form, and hence be comfortably administrable in consistent, accurate dosages. The compositions of the invention containing DuraSite® remain in place for prolonged periods of time to provide sustained release of the ophthalmic medicament.

As used herein the term “retained in or carried with” or “retaining or carrying” embraces generally all ways that bromfenac can be associated with the flowable mucoadhesive polymer. For example, bromfenac can be in aqueous solution dispersed throughout the polymer. A bromfenac concentration of up to about 0.36% will be in solution mixed with or dispersed throughout the flowable mucoadhesive polymer carrier.

Bromfenac can also be in suspension with the polymer depending on its concentration. For example, when bromfenac is used in an amount more than about 0.36% by weight of the composition, some of the bromfenac can be in suspension with the polymer carrier while an amount of up to about 0.13% of bromfenac will still be in solution and mixed with the polymer carrier.

As used herein the term “sustained release delivery system” or “sustained release composition” refers to a composition comprising a flowable mucoadhesive polymer—which is a carboxy-containing polymer such as polycarbophil and DuraSite®, as described in U.S. Pat. No. 5,192,535 which facilitates a sustained release of bromfenac. Such compositions may include other biologically active agents besides bromfenac. In some embodiments, the sustained release compositions of the invention can contain from about 0.01% (w/w) to about 0.05% of bromfenac (free acid). In one embodiment, the range of bromfenac loading is between about 0.01% (w/w) to about 0.04%. In another embodiment, the range of bromfenac loading is between about 0.02% (w/w) to about 0.04%. The sustained release delivery systems or compositions of this invention can be formed into many formulations or shapes such as a solution, a gel, a film, a pellet, a rod, a filament, a cylinder, a disc, a wafer, nanoparticles or a microparticle. A “microparticle” as defined herein, comprises a blend polymer component having a diameter of less than about one millimeter and having bromfenac dispersed therein. A microparticle can have a spherical, non-spherical or irregular shape. Typically, the microparticle will be of a size suitable for injection. In one embodiment, the size range for microparticles is from about one to about 25 microns in diameter.

The composition is typically applied up to 2 times a day, but may be applied more often as needed. As defined herein, a sustained release of a biologically active agent is a release of the biologically active agent (e.g., bromfenac) from a sustained release delivery system or composition. The release occurs over a period which is longer than that period during which a therapeutically significant amount of the biologically active agent would be available following direct administration of a solution of the biologically active agent. In one embodiment, a sustained release of biologically active agent occurs over a period of greater than 4-6 hours. Sustained release can be a continuous or a discontinuous release, with relatively constant or varying rates of release. The continuity of release and level of release can be affected by the type of polymer composition used (e.g., monomer ratios, molecular weight, and varying combinations of polymers), agent loading, and/or selection of excipients to produce the desired effect.

As used herein the term “treating” or “treatment” refers to reducing, ameliorating reversing, alleviating, inhibiting the progress of, or preventing the signs and symptoms of ocular surface allergies. The term also encompasses prophylaxis, therapy and cure. The subject receiving “treatment,” or whom undergoes “treating” is any mammal in need of such treatment for (ocular surface allergies), including primates, in such as humans, and other mammals such as equines, cattle, swine and sheep; and poultry and domesticated mammals and pets in general.

The term “therapeutically effective amount” as used herein means that the amount of a composition elicits a beneficial biological or medicinal response in a tissue, system, animal or human. For example, a therapeutically effective amount of a composition of the invention is a dose which leads to a clinically detectable improvement or treatment (as defined above) of the eye of a subject suffering from the signs and symptoms of ocular surface allergies.

As used herein, the term “about” refers to an approximation of a stated value within an acceptable range, e.g. +/−5% of the stated value.

It is an aspect of the present invention to provide novel ophthalmic compositions for treating ocular surface allergies; such compositions include bromfenac and a flowable mucoadhesive polymer to increase the retention of bromfenac in the eye for a longer period of time.

It is a further aspect of the invention to provide a novel sustained release delivery system for bromfenac, which includes bromfenac and a flowable mucoadhesive polymer. It is an object of this invention to provide a novel sustained release delivery system for topical ophthalmic delivery of bromfenac.

A further aspect of this invention is to provide novel bromfenac compositions and sustained release ophthalmic delivery systems suitable for administration at intervals of once or twice a day.

A still further aspect of this invention is to provide novel methods for treating, ameliorating or reducing ocular surface allergies by providing a composition or a delivery system containing bromfenac and a flowable mucoadhesive polymer, which has a prolonged release time for bromfenac, while facilitating a high absorption and retention of bromfenac by the eye over the release time period.

Yet another aspect of this invention is to provide novel methods for treating, ameliorating or reducing ocular surface allergies by providing a composition or a delivery system containing a low dosage of bromfenac and a flowable mucoadhesive polymer.

Bromfenac is a non-steroidal anti-inflammatory agent which has the structural formula of

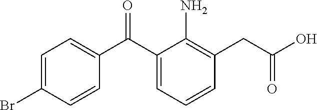 [SMILES: Nc1c(CC(=O)O)cccc1C(=O)c1ccc(Br)cc1]

The above compound to be used in accordance with the invention may be in a salt form or a hydrated form or both. The salt forms include alkali metal salts such as sodium salt and potassium salt, alkaline earth metal salts such as calcium salt and magnesium salt, among others, and any salt may suitably be used, provided that it can attain the object of the invention. The hydrated forms include monohydrate, sessquihydrate (1.5H₂O), dihydrate, pentahydrate and any other hydrate forms may suitably be used, provided that it can attain the object of the invention.

The present invention also provides kits including a composition having bromfenac and a flowable mucoadhesive polymer for application to the eye of a mammal. The kit further includes instructions for how use the composition, eye dropper and other useful paraphanalia for topical delivery to the eye.

In one embodiment, according to any of the above aspects of the invention, the composition of the invention has a pH of about 8.0 to about 8.6; in other embodiments the pH is about 8.3.

In one embodiment, according to any of the above aspects of the invention, the viscosity of the compositions of the invention is in the range of about 1,000 to about 2,000 cps.

In another embodiment, the viscosity of the composition of the invention is about 1,500 cps. When formulated as a topical ophthalmic delivery system, the viscosity of the composition of the invention is desirably in a range suitable for administration to the eye in drop form, such as a viscosity from about 1,000 to about 2,000 cps. In one embodiment, according to any of the above aspects of the invention, the bromfenac is retained in or carried with the flowable mucoadhesive polymer. The flowable mucoadhesive polymer increases the retention of bromfenac in the eye for a longer period of time. In another embodiment, the entire bromfenac content of the composition of the invention is in aqueous solution. Bromfenac formulated in DuraSite has been shown in pharmacokinetic experiments to give enhanced levels 2 to 4 times in ocular tissues over standard eyedrop formulations.

In one embodiment, according to any of the above aspects of the invention, a percentage of bromfenac content of the compositions of the invention is in aqueous solution with the polymer while the remaining bromfenac remains in suspension with the polymer.

In another embodiment, the flowable mucoadhesive polymers of the invention are crosslinked carboxy-vinyl polymers as carboxy-containing polymers. Suitable carboxy-containing polymers for use in the present invention and method for making them are described in U.S. Pat. No. 5,192,535 to Davis et al. These polymer carriers include lightly crosslinked carboxy-containing polymers such as polycarbophil, or Carbopols®, dextran, cellulose derivatives, polyethylene glycol 400 and other polymeric demulcents such as polyvinylpyrolidone, polysaccaride gels and Gelrite®. In another embodiment, a carboxy-containing polymer system known by the tradename DuraSite® is used. DuraSite® is a lightly crosslinked polymer containing polycarbophil which is a sustained release topical ophthalmic delivery system that releases the drug at a controlled rate.

The lightly crosslinked polymers of acrylic acid or the like used in practicing this invention are, in general, well known in the art. In one embodiment such polymers are ones prepared from at least about 90% or from about 95% to about 99.9% by weight, based on the total weight of monomers present, of one or more carboxyl-containing monoethylenically unsaturated monomers. Acrylic acid is a carboxyl-containing monoethylenically unsaturated monomer, but other unsaturated, polymerizable carboxyl-containing monomers, such as methacrylic acid, ethacrylic acid, .beta.-methylacrylic acid (crotonic acid), cis-.alpha.-methylcrotonic acid (angelic acid), trans-.alpha.-methylcrotonic acid (tiglic acid), .alpha.-butylcrotonic acid, .alpha.-phenylacrylic acid, .alpha.-benzylacrylic acid, .alpha.-cyclohexylacrylic acid, .beta.-phenylacrylic acid (cinnamic acid), coumaric acid (o-hydroxycinnamic acid), umbellic acid (p-hydroxycoumaric acid), and the like can be used in addition to or instead of acrylic acid.

Such polymers are crosslinked by using a small percentage, i.e., less than about 5%, such as from about 0.5% or from about 0.1% to about 5%, or from about 0.2% to about 1%, based on the total weight of monomers present, of a polyfunctional crosslinking agent. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N, N-diallylacrylamide; N, N-diallylmethacrylamide and the like. Also included are polyalkenyl polyether crosslinking agents containing two or more alkenyl ether groupings per molecule, or alkenyl ether groupings containing terminal H₂C═C<groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown U.S. Pat. No. 2,798,053, which incorporated herein by reference in its entirety. Diolefinic non-hydrophilic macromeric crosslinking agents having molecular weights of from about 400 to about 8,000, such as insoluble di- and polyacrylates and methacrylates of diols and polyols, diisocyanate-hydroxyalxyl acrylate or methacrylate reaction products, and reaction products of isocyanate terminated prepolymers derived from polyester diols, polyether diols or polysiloxane diols with hydroxyalkylmethacrylates, and the like, can also be used as the crosslinking agents; see, e.g., Mueller et al. U.S. Pat. Nos. 4,192,827 and 4,136,250, which incorporated herein by reference in its entirety.

The lightly crosslinked polymers can of course be made from a carboxyl-containing monomer or monomers as the sole monoethylenically unsaturated monomer present, together with a crosslinking agent or agents. They can also be polymers in which up to about 40%, or from about 0% to about 20% by weight, of the carboxyl-containing monoethylenically unsaturated monomer or monomers has been replaced by one or more non-carboxyl-containing monoethylenically unsaturated monomers containing only physiologically and ophthalmologically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like; see Mueller et al. U.S. Pat. No. 4,548,990, which incorporated herein by reference in its entirety, for a more extensive listing of such additional monoethylenically unsaturated monomers. In one embodiment, polymers are lightly crosslinked acrylic acid polymers wherein the crosslinking monomer is 2,3-dihydroxyhexa-1,5-diene or 2,3-dimethylhexa-1,5-diene.

The lightly crosslinked polymers used in practicing this invention are prepared by suspension or emulsion polymerizing the monomers, using conventional free radical polymerization catalysts, to a dry particle size of not more than about 25 μm in equivalent spherical diameter; e.g., to provide dry polymer particles ranging in size from about 1 to about 25 μm, or from about 1 to about 5 μm, in equivalent spherical diameter. In general, such polymers will range in molecular weight estimated to be about 400,000 to 4,000,000,000 Da.

According to any of the above aspects of the invention, the composition of the invention is an aqueous mixture that can also contain amounts of suspended lightly crosslinked polymer particles ranging from about 0.5% to about 1.5% by weight, or from about 0.8% to about 1.0% by weight, based on the total weight of the aqueous mixture. The aqueous mixture can be an aqueous solution of bromfenac and a flowable mucoadhesive polymer or an aqueous suspension of bromfenac and a flowable mucoadhesive polymer. In certain embodiments, the composition of the invention is prepared using pure, sterile water, such as deionized or distilled, having no physiologically or ophthalmologically harmful constituents, and is adjusted to a pH of from about 8.0 to about 8.6, in some embodiments from about 8.2 to about 8.4, and in other embodiments to a pH of about 8.3 using any physiologically and ophthalmologically acceptable pH adjusting acid, base or buffer, e.g., acids such as acetic, boric, citric, lactic, phosphoric, hydrochloric, or the like, bases such as sodium hydroxide, sodium phosphate, sodium borate, sodium citrate, sodium acetate, sodium lactate, THAM (trishydroxymethylamino-methane), or the like and salts and buffers such as citrate/dextrose, sodium bicarbonate, ammonium chloride and mixtures of the aforementioned acids and bases. For example, bromfenac or its salt at may be dissolved and added by sterile filtration to a preparation containing sodium chloride, DuraSite® and surfactant. This mixture may then be adjusted to the appropriate pH by known techniques, for example by the addition of sodium hydroxide. Other methods will be apparent to one skilled in the art.

The well established soothing effect of Durasite® may contribute to the positive effect of the present formulations for treating ocular surface allergies. Moreover, Durasite® extends surface coverage in the eye and also contributes to lowering the allergen(s)' contact time and surface contact with the eye, thereby achieving an additional therapeutic effect.

In another aspect, the invention relates to a composition or method for combination therapy of the eye of a mammal including an ophthalmic composition having a therapeutically effective amount of bromfenac, a therapeutically effective amount of a vasoconstrictor or combination of vasoconstrictors and a flowable mucoadhesive polymer such as DuraSite®. In general, vasodilator (or vasoconstrictor; the terms are interchangeable) is included in the compositions at a range of from about 0.01 to about 0.25 wt % of the composition. Nonlimiting examples of the vasoconstrictor are ephedrine hydrochloride, which is included in some embodiments at a concentration of from 0.095 to 0.130%, and in other embodiments at a concentration of about 0.123% of the formulation; naphazoline hydrochloride, which is used in certain embodiments at a a concentration of about 0.01 to 0.03 wt % of the formulation; phenylephrine hydrochloride, which may be used in certain embodiments at a concentration of about 0.08 to 0.2 wt % of the formulation; and tetrahydrozoline hydrochloride, which in certain embodiments is used at a concentration of 0.01 to 0.05% of the formulations of the invention.

When formulating the composition of the invention as either an aqueous solution or an aqueous suspension, the osmolality can be adjusted to from about 10 mOsm/kg to about 400 mOsm/kg, using appropriate amounts of physiologically and ophthalmologically acceptable salts. Sodium chloride approximates physiologic fluid, and amounts of sodium chloride ranging from about 0.01% to about 1% by weight, or from about 0.05% to about 0.45% by weight, based on the total weight of the aqueous suspension, provide osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can also be used in addition to or instead of sodium chloride to achieve osmolalities within the above-stated ranges. Sugars like mannitol, dextrose, glucose or other polyols may be added to adjust the osmolality.

The amounts of flowable mucoadhesive polymer, the pH, and the osmotic pressure chosen from within the above-stated ranges are correlated with one another and with the degree of crosslinking of the polymer to give aqueous solutions or suspensions having viscosities ranging from about 1,000 to about 2,000 or 5,000 to about 20,000 cps respectively, as measured at room temperature (about 25° C.) using a Brookfield Digital LVT Viscometer equipped with a number 25 spindle and a 13R small sample adapter at 12 rpm. The compositions of the present invention have a viscosity that is suited for the selected route of administration. Alternatively, the viscosity can be 1000 to 3400 cps as measured with a Brookfield cone and plate viscosity DV-II+CP with the spindle No. CP-52 at 6 rpm.

In one embodiment, according to any of the above aspects of the invention, the compositions of the present invention ordinarily contain one or more surfactants and, if desired, one or more adjuvants, including additional medicaments, buffers, antioxidants, tonicity adjusters, preservatives, thickeners or viscosity modifiers, and the like. Additives in the formulation may desirably include sodium chloride, EDTA (disodium edetate), and BAC (benzalkonium chloride) or sorbic acid, or both. The formulations can also be prepared non-preserved.

Compositions delivered by means of the sustained release medicament delivery system of this invention typically have residence times in the eye ranging from about 2 to about 6 hours. The bromfenac contained in these compositions is released from the composition at rates that depend on such factors as bromfenac itself and its physical form, the extent of drug loading and the pH of the system, as well as on any drug delivery adjuvants, such as ion exchange resins compatible with the ocular surface, which may also be present in the composition. In one embodiment, according to any of the aspects of the present invention, the composition of the invention provides a sustained concentration of bromfenac of between 10⁻⁶ and 10⁻⁸ M in the aqueous or treated tissue of the eye for at least two hours, and in certain embodiments, at least three hours. In another embodiment, the composition of the invention provides sustained concentration of bromfenac of between 10⁻⁶ and 10⁻⁸ M, in the aqueous or treated tissue of the eye for at least two hours, or at least three hours. In order that those skilled in the art can more fully appreciate aspects of this invention, the following Tables and examples are set forth. These examples are given solely for purposes of illustration and should not be considered as expressing limitations.

### EXAMPLE 1

Polycarbophil (Noveon® AA-1) was slowly dispersed into a citrate buffer solution containing dissolved EDTA and sodium chloride at approximately 50% of the final batch size. The resulting dispersion, which had a pH of a bout 3.0 to 3.5, was stirred with an overhead stirrer until visibly well hydrated. The mixture was sterilized by autoclaving at 121° C. for 20 minutes. The pH was then brought up to approximately 4.0 to 4.4 with 2N sodium hydroxide. Bromfenac sodium was dissolved in a mannitol solution containing dissolved benzalkonium chloride and Poloxamer 407 at approximately 20% of the final batch size. The resulting solution was then sterile filtered (0.22 μm filter) in to the polymer dispersion and stirred for 10 minutes. The pH of the bromfenac-polymer dispersion was brought to 8.3 with 2N sodium hydroxide. Sterile make up water was added by sterile filtration to the formulation to final weight and mixed for at least 5 minutes. The formulation was aseptically filled into 5 ml bottles. See Table 1 for formulation composition.

TABLE 1
Composition of Bromfenac Formulations Preserved

| | Concentration (% w/w) | | |
|---|---|---|---|
| Excipient | Low Strength | Mid Strength | High Strength |
| Bromfenac | 0.01% | 0.02 | 0.04 |
| Polycarbophil | 0.925 | 0.925 | 0.925 |
| Citric acid | 0.2 | 0.2 | 0.2 |
| Sodium citrate | 0.14 | 0.14 | 0.14 |
| Sodium chloride | 0.27 | 0.27 | 0.27 |
| Poloxamer 407 | 0.2 | 0.2 | 0.2 |
| Boric acid | 0.49 | 0.49 | 0.49 |
| Sodium borate | 0.51 | 0.51 | 0.51 |
| Benzalkonium | 0.005 | 0.005 | 0.005 |
| Chloride | | | |
| Sodium hydroxide, 2N | q.s. to pH 8.3 | q.s. to pH 8.3 | q.s. to pH 8.3 |
| Water | q.s. to 100% | q.s. to 100% | q.s. to 100% |

### EXAMPLE 2

TABLE 2
Composition of Bromfenac Formulations Non-Preserved

| | Concentration (% w/w) | | |
|---|---|---|---|
| Excipient | Low Strength | Mid Strength | High Strength |
| Bromfenac | 0.01% | 0.025 | 0.05 |
| Polycarbophil | 0.91 | 0.91 | 0.91 |
| Citric acid | 0.2 | 0.2 | 0.2 |
| Sodium citrate | 0.025 | 0.025 | 0.025 |
| Sodium chloride | 0.1 | 0.1 | 0.1 |
| Poloxamer 407 | 0.2 | 0.2 | 0.2 |
| Mannitol | 1.0 | 1.0 | 1.0 |
| Boric Acid | 0.49 | 0.49 | 0.49 |
| Sodium Borate | 0.51 | 0.51 | 0.51 |
| Sodium hydroxide, 2N | q.s. to pH 8.3 | q.s. to pH 8.3 | q.s. to pH 8.3 |
| Water | q.s. to 100% | q.s. to 100% | q.s. to 100% |

TABLE 3
Compositons with Bromfenac and Vasodilator

| | Concentration (% w/w) | | |
|---|---|---|---|
| Excipient | Low Strength | Mid Strength | High Strength |
| Bromfenac | 0.01% | 0.025 | 0.05 |
| ephedrine | 0.095 | 0.115 | 0.03 |
| hydrochloride* | | | |
| naphazoline | 0.01 | 0.02 | 0.03 |
| hydrochloride* | | | |
| tetrahydrozoline | 0.01 | 0.025 | 0.05 |
| hydrochloride* | | | |
| phenylephrine | 0.08 | 0.15 | 0.2 |
| hydrochloride* | | | |
| Polycarbophil | 0.91 | 0.91 | 0.91 |
| Citric acid | 0.2 | 0.2 | 0.2 |
| Sodium citrate | 0.025 | 0.025 | 0.025 |
| Sodium chloride | 0.1 | 0.1 | 0.1 |
| Poloxamer 407 | 0.2 | 0.2 | 0.2 |
| Mannitol | 1.0 | 1.0 | 1.0 |
| Boric Acid | 0.49 | 0.49 | 0.49 |
| Sodium Borate | 0.51 | 0.51 | 0.51 |
| Sodium hydroxide, 2N | q.s. to pH 8.3 | q.s. to pH 8.3 | q.s. to pH 8.3 |
| Water | q.s. to 100% | q.s. to 100% | q.s. to 100% |

*The compositions in Table 3 contain a single vasodilator selected from the four listed vasodilators. Alternatively, a combination of vasodilators may be included in the composition in the ranges provided for each in Table 3, but the total amount of vasodilator in the composition should not exceed 0.25 wt % of the composition.

Compositions according to Tables 2 were made as follows. Polycarbophil (Noveon® AA-1) was slowly dispersed into a citrate buffer solution containing dissolved EDTA and sodium chloride at approximately 50% of the final batch size. The resulting dispersion, which had a pH of a bout 3.0 to 3.5, was stirred with an overhead stirrer until visibly well hydrated. The mixture was sterilized by autoclaving at 121° C. for 20 minutes. The pH was then brought up to approximately 4.0 to 4.4 with 2N sodium hydroxide. Bromfenac sodium was dissolved with boric acid, sodium borate and Poloxamer 407 at approximately 20% of the final batch size. The resulting solution was then sterile filtered (0.22 μm filter) in to the polymer dispersion and stirred for 10 minutes. The pH of the bromfenac-polymer dispersion was brought to 8.3 with 2N sodium hydroxide. Sterile make up water was added by sterile filtration to the formulation to final weight and mixed for at least 5 minutes. The formulation was aseptically filled into unit dose containers and over wrapped with an aluminum foil laminate.

In certain embodiments, a similar process is used to prepare a formulation containing a vasodilator or combination of vasodilators, such as those listed in Table 3.

TABLE 4
Compositions and Target Attributes of NSAID Formulations

| | Concentration (% w/w) | | |
|---|---|---|---|
| | 0.01% BF - | 0.02% BF - | |
| Excipient | DuraSite | DuraSite | 0.04% BF DuraSite |
| Bromfenac | 0.01 | 0.02 | 0.04 |
| Citric acid | 0.2 | 0.2 | 0.2 |
| Sodium citrate | 0.14 | 0.14 | 0.14 |
| dihydrate | | | |
| Edetate disodium | 0.1 | 0.1 | 0.1 |
| dihydrate | | | |
| Sodium chloride | 0.025 | 0.025 | 0.025 |
| Poloxamer 407 | 0.2 | 0.2 | 0.2 |
| Boric Acid | 0.49 | 0.49 | 0.49 |
| Sodium Borate | 0.51 | 0.51 | 0.51 |
| 2N Sodium hydroxide | q.s. to | q.s. to | q.s. to pH 8.3 |
| | pH 8.3 | pH 7.4 | |
| Water | q.s. to 100% | q.s. to 100% | q.s. to 100% |

| | Target Value | | | | | |
|---|---|---|---|---|---|---|
| | 0.01% BF - | | 0.02% KT - | | 0.04% BF - | |
| Attribute | DuraSite | | DuraSite | | DuraSite | |
| Bromfenac | 0.01 | | 0.02 | | 0.04 | |
| pH | 8.3 | | 8.3 | | 8.3 | |
| Viscosity | 1500 | cps | 1500 | cps | 1500 | cps |
| Osmolality | 240 | mOsm/kg | 240 | mOsm/kg | 240 | mOsm/kg |

The embodiments within the specification provide an illustration of embodiments of the invention and should not be construed to limit the scope of the invention. The skilled artisan readily recognizes that many other embodiments are encompassed by the invention.

We claim:

1. An ophthalmic composition for the treatment of ocular surface allergies comprising bromfenac in an amount of 0.01% to 0.05% by weight of the composition and a flowable mucoadhesive polymer, wherein the composition has a viscosity formulated for administration to the eye of a mammal in drop form.

2. The ophthalmic composition according to claim 1, wherein the flowable mucoadhesive polymer is a crosslinked carboxy-containing polymer.

3. The ophthalmic composition according to claim 2, wherein the crosslinked carboxy-containing polymer is polycarbophil.

4. The ophthalmic composition according to claim 1, wherein the flowable mucoadhesive polymer is in an amount of about 0.5% to about 1.5% by weight of the composition.

5. The ophthalmic composition according to claim 1, wherein the bromfenac is in an amount of 0.01% to 0.04% by weight of the composition.

6. The ophthalmic composition according to claim 1, wherein the composition has a pH about 8.0 to 8.6.

7. The ophthalmic composition according to claim 1, wherein the viscosity of the composition is in the range of about 1,000 to about 2,000 cps.

8. The ophthalmic composition according to claim 1 further comprising a vasodilator.

9. The ophthalmic composition according to claim 8 wherein the vasodilator is selected from the group consisting of ephedrine hydrochloride, naphazoline hydrochloride, phenylephrine hydrochloride and tetrahydozoline hydrochloride.

10. A sustained release bromfenac delivery system, comprising a flowable mucoadhesive polymer and a therapeutically effective amount of bromfenac in an ophthalmic composition;
wherein the flowable mucoadhesive polymer is in an amount of about 0.5% to about 1.5% by weight of the composition and bromfenac is in an amount of about 0.005% to about 0.5% by weight of the composition.

11. The sustained release bromfenac delivery system according to claim 10, wherein the flowable mucoadhesive polymer is a carboxy containing polymer.

12. The sustained release bromfenac delivery system according to claim 11, wherein the carboxy containing polymer is polycarbophil.

13. The sustained release bromfenac delivery system according to claim 10, wherein the composition has a pH of about 8.0 to 8.6.

14. The sustained release bromfenac delivery system according to claim 10, wherein the viscosity of the composition is in the range of about 1,000 to about 2,000 cps.

15. The sustained release bromfenac delivery system according to claim 10 further comprising a vasodilator.

16. A method of treating ocular surface allergies, comprising the steps of:
(a) providing an ophthalmic composition comprising bromfenac in an amount of about 0. 01% to about 0.5% by weight of the composition and a flowable mucoadhesive polymer in an amount of about 0.5% to about 1.5% by weight of the composition;
(b) administering said composition to the eye of a mammal in need thereof to treat ocular surface allergies of the eye.

17. The method for treating ocular surface allergies according to claim 16, wherein the ocular surface allergy is caused by a seasonal allergen selected from the group consisting of pollen, ragweed, trees, grass, and mold.

18. The method for treating ocular surface allergies according to claim 16, wherein the flowable mucoadhesive polymer is a crosslinked carboxy-containing polymer.

19. The method for treating ocular surface allergies according to claim 18, wherein the crosslinked carboxy-containing polymer is polycarbophil.

20. The method for treating ocular surface allergies according to claim 16 wherein, the composition is administered once a day.

21. The method for treating ocular surface allergies according to claim 16, wherein the composition has a pH of about 8.0 to 8.6.

22. The method for treating ocular surface allergies according to claim 16, wherein the viscosity of the composition is in the range of about 1,000 to about 2,000 cps.

23. The method for treating ocular surface allergies according to claim 16, wherein the bromfenac is in an amount of 0.01% to 0.04% by weight of the composition.

24. The method for treating ocular surface allergies according to claim 16, wherein the ophthalmic composition further comprises a vasodilator.
